(19) United States
(12) Patent Application Publication
(10) Pub. No.: US 2007/0038196 A1
(43) Pub. Date: Feb. 15, 2007

(54) Absorbent structure and absorbent article comprising the absorbent structure

(75) Inventors: Karl Karlsson, Goteborg (SE); Eje Osterdahl, Vastra Frolunda (SE); Charlotta Hanson, Goteborg (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 11/482,161

(22) Filed: Jul. 7, 2006

Related U.S. Application Data
(63) Continuation-in-part of application No. 11/025,060, filed on Dec. 30, 2004, now abandoned.
(60) Provisional application No. 60/532,951, filed on Dec. 30, 2003.

(51) Int. Cl. A61F 13/15

(52) U.S. Cl. 604/368; 604/374; 604/378

(57) ABSTRACT

An absorbent structure, for use in a diaper, an incontinence pad, a sanitary article or the like, has at least one absorbent layer including fluff pulp and superabsorbent particles. The average absorption capacity per superabsorbent particle in the absorbent layer is greater than 8.0 mg sodium chloride solution, and the number of superabsorbent particles per cm³ of the absorbent layer is smaller than 1100. A diaper, an incontinence pad, a sanitary article or the like includes this absorbent structure.

### CROSS-REFERENCE TO RELATED APPLICATION

The present application is a continuation-in-part of U.S. application Ser. No. 11/025,060, filed Dec. 30, 2004, which claims the benefit of U.S. Provisional Application No. 60/532,951 filed in the United States on Dec. 30, 2003, the entire contents of which is hereby incorporated by reference.

### TECHNICAL FIELD

The present invention relates to an absorbent structure for use in an absorbent article such as a diaper, an incontinence pad, a sanitary towel or the like, which absorbent structure has at least one absorbent layer comprising fluff pulp and superabsorbent particles.

### BACKGROUND OF THE INVENTION

An absorbent structure for disposable absorbent articles such as diapers, incontinence pads and sanitary towels is usually constructed from one or more layers of hydrophilic fibres, for example cellulose fluff pulp. In order to obtain high absorption capacity and also a high liquid-retaining capacity when the article is subjected to external loading, the absorbent structure usually contains superabsorbent particles, which are polymers with the ability to absorb many times their own weight of water or body fluid. The effectiveness of the superabsorbent depends on many factors such as, for example, the physical shape of the superabsorbent particles. Other examples of properties which influence the functioning of the superabsorbent are absorption rate, gel strength and liquid-retaining capacity.

The absorbent structure can also contain other components, for example in order to improve its liquid-spreading properties or increase its cohesive capacity and ability to withstand deformation during use.

It is of considerable importance that the absorbent article is capable of rapidly receiving and absorbing large quantities of liquid. It is also of considerable importance that the total absorption capacity of the article can be utilized. In order for it to be possible to utilize the total absorption capacity of the article, it is desirable that the liquid can be spread from the wetting area to other parts of the absorbent structure.

One problem, above all for diapers and incontinence pads which are intended to receive and absorb relatively large quantities of liquid, is that there is a risk of them leaking before their total absorption capacity is fully utilized. One cause of leakage is that the absorbent structure, in particular when repeated wetting takes place, has an impaired ability to receive and absorb large quantities of liquid rapidly. A major cause of it being difficult for the absorbent structure to function satisfactorily when repeated wetting takes place, that is to say a second wetting and subsequent wettings, is that the superabsorbent material in a swollen state can block the pores in the porous fibrous structure and thus interfere with the transport of liquid from the wet area out to other parts of the absorbent structure. This phenomenon is referred to as “gel blocking” and results in the total absorption capacity of the absorbent structure not being utilized optimally. It also leads to an increased risk of leakage.

The problem of gel blocking increases when the proportion of superabsorbent material in an absorbent structure is high. In order to obtain an article which is discreet and comfortable to wear, however, it is an advantage to have a thin article which contains a relatively high proportion of superabsorbent material.

### SUMMARY OF THE INVENTION

The problem of gel blocking during use of thin absorbent articles having a relatively high content of superabsorbent material has been reduced by means of the present invention.

An absorbent structure according to a preferred embodiment of the present invention is characterized mainly in that the average absorption capacity per superabsorbent particle in the absorbent layer is greater than 8.0 mg and in that the number of superabsorbent particles per cm³ of the absorbent layer is smaller than 1100. The absorption capacity is measured using 0.9% by weight sodium chloride solution.

By limiting the number of superabsorbent particles per unit of volume, it has been found that it is possible to maintain a fibrous network with a pore structure which can transport liquid in the absorbent structure even after the structure has been subjected to a first wetting. It has also been found that, with a limited number of superabsorbent particles per unit of volume, it is preferred that the average absorption capacity per superabsorbent particle is greater than 8.0 mg. The advantage of such an absorbent structure is that the risk of gel blocking decreases at the same time as it is possible to obtain a thin absorbent structure.

According to a preferred embodiment of the present invention, the average absorption capacity per superabsorbent particle in the absorbent layer is greater than 9.5 mg. The absorption capacity is measured using 0.9% by weight sodium chloride solution. Furthermore, the number of superabsorbent particles per cm³ of the absorbent layer is smaller than 600.

According to another preferred embodiment, the average absorption capacity per superabsorbent particle in the absorbent layer is greater than 14.0 mg. The absorption capacity is measured using 0.9% by weight sodium chloride solution. In such an embodiment, the number of superabsorbent particles per cm³ of the absorbent layer is smaller than 450.

According to a further preferred embodiment, the superabsorbent particles have a particle size which is greater than 600 μm. The superabsorbent particles are preferably polyacrylate-based. In order to obtain a high absorption capacity, it is also possible to change the morphology of the superabsorbent particles. An example of superabsorbent particles with a changed morphological structure is microporous superabsorbent particles. A high absorption capacity can also be obtained by means of a special chemical composition of the superabsorbent particles.

The superabsorbent particles can be surface cross-linked or have a gradually increasing cross-linking towards the surface of the particles. A surface cross-linked superabsorbent is cross-linked in two different steps. First, the polymer is cross-linked so that a homogeneous cross-linked gel is formed. In cases where polymerization and cross-linking do not result in particles being formed simultaneously, particles are produced in a following process step. In another following process step, the formed particles are cross-linked in the second step, but then only partly. The additional cross-linking can be effected so that there is a higher cross-linker content next to the surface of the particle compared with the centre of the particle. In this way, a more firmly cross-linked particle shell is produced, which surrounds a particle core with a lower degree of cross-linking.

Superabsorbents with a low degree of cross-linking provide a high absorption capacity. However, a problem with such superabsorbents is that, in a swollen state, they are soft and sticky, which results in the risk of gel blocking in the absorbent structure already being high at a low superabsorbent material content. Superabsorbents with a high degree of cross-linking keep their shape better in a swollen state and do not stick to the same great extent either. However, a problem with a superabsorbent with a high degree of cross-linking is that it has a considerably lower absorption capacity. So, by surface cross-linking the superabsorbent, or alternatively creating a cross-linking gradient so that the particle surface is cross-linked more firmly than the inner particle core, a superabsorbent is obtained which has both high absorption capacity and essentially maintains its shape in a swollen state.

According to a preferred embodiment of an absorbent structure according to the present invention, the average distance between centres of the superabsorbent particles in the absorbent layer in a dry state is greater than 700 micrometres, more preferably greater than 1000 micrometres, and even more preferably greater than 1200 micrometres. The average centre-centre distance (I_{cc}) of the superabsorbent particles is obtained using the following equation:
I_{cc}=(1/n)^{1/3}

n=number of superabsorbent particles per unit of volume of material

According to another preferred embodiment, the density of the absorbent layer is greater than 0.12 g/cm³, more preferably greater than 0.17 g/cm³ and even more preferably greater than 0.25 g/cm³. The absorbent layer can moreover comprise bonding means, such as bonding fibres for example. Examples of bonding fibres are synthetic fibres made of polyolefin. In order to function as bonding fibres, the fibres are heated to their melting point, the fibres being bonded to the material in the absorbent layer. Bonding fibres made of bicomponent fibres are common. If bicomponent fibres are used as bonding fibres, one component is melted while the other component is intact, that is to say does not melt but instead maintains the structure of the fibre.

The invention also relates to an absorbent article such as a diaper, an incontinence pad, a sanitary towel or the like, which comprises an absorbent structure according to any one of the embodiments described.

### BRIEF DESCRIPTION OF THE FIGURES

FIG. 1 shows a diaper according to a preferred embodiment of the present invention, seen from the side which is intended to lie against the wearer during use;

FIG. 2 shows a cross section along the line II-II through the diaper shown in FIG. 1;

FIG. 3 shows a cross section of an alternative preferred embodiment of an absorbent article according to the present invention.

### DETAILED DESCRIPTION OF THE PREFERRED EMBODIMENTS

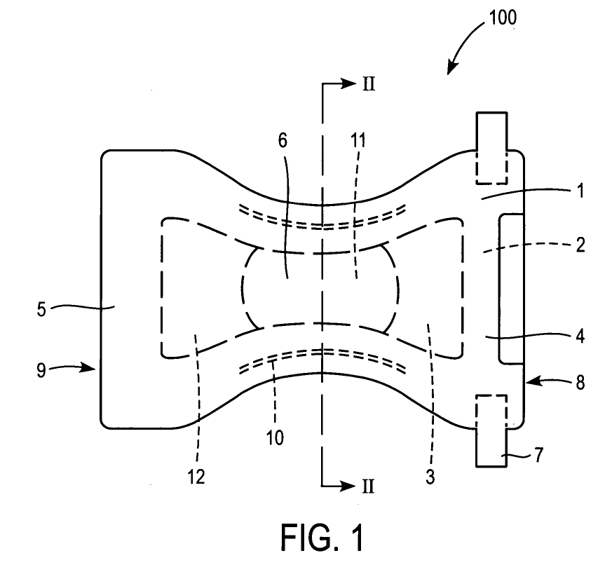

The diaper 100 shown in FIG. 1 comprises a liquid-permeable surface layer 1, a backing layer 2, which is at least essentially liquid-impermeable, and an absorbent structure 3 enclosed between the liquid-permeable surface layer 1 and the backing layer 2.

The diaper is intended to surround the lower part of the abdomen of a wearer like a pair of absorbent underpants. To this end, it is shaped with a rear portion 4 and a front portion 5, and a narrower crotch portion 6 which is located between the front portion 5 and the rear portion 4 and is intended during use to be arranged in the crotch of the wearer between the legs of the latter. In order that it is possible for the diaper to be fastened together in the desired pants-shape, tape tabs 7 are arranged close to the rear waist edge 8 of the diaper. During use, the tape tabs 7 are fastened to the front portion 5 of the diaper, close to the front waist edge 9, so that the diaper is held together around the waist of the wearer. Other fastening devices are of course also possible, such as hook and loop fastening for example.

The diaper 100 according to FIG. 1 also comprises pretensioned elastic means 10 which may include elastic bands, thread-covered elastic threads, elastic foam or another suitable material. For the sake of simplicity, the elastic means 10 have in FIG. 1 been shown in the stretched state. As soon as stretching stops, however, they contract and form elastic leg-bands of the diaper.

The liquid-permeable surface layer 1 is, for example, a nonwoven material or a perforated film, or a laminate thereof. Examples of polymers from which the liquid-permeable surface layer 1 can be made are polyethylene, polypropylene, polyester or copolymers thereof. In order that the liquid-permeable surface layer 1 will allow the discharged body fluid to pass through rapidly, it is common for the surface layer to be surfactant-coated and/or perforated. Another suitable material for use as the liquid-permeable surface layer is a layer of continuous fibres which are interconnected in a spot, line or patch bonding pattern but are otherwise on the whole not bonded to one another. The backing layer 2 is, for example, a plastic film, which is preferably breathable, a hydrophobic nonwoven layer or a laminate thereof.

The absorbent structure 3 of the diaper 100 is preferably constructed from an upper liquid-receiving layer 11 and a lower liquid-distribution and storage layer 12. The lower liquid-distribution and storage layer 12 has a greater extent in the plane of the article than the upper liquid-receiving layer 11. The upper receiving layer 11 is to be capable of rapidly receiving large quantities of liquid in a short time, that is to say have a high instantaneous liquid absorption capacity, while the lower distribution and storage layer 12 is to have a high wicking capacity and high storage capacity and to be capable of draining liquid from the receiving layer 11. The lower distribution and storage layer 12 in the absorbent structure 3 includes an absorbent layer according to a preferred embodiment of the present invention. The lower liquid-distribution and storage layer 12 therefore also comprises superabsorbent particles in addition to cellulose fluff pulp. The average absorption capacity per superabsorbent particle in the liquid-distribution and storage layer 12 is preferably greater than 8.0 mg sodium chloride solution. Furthermore, the number of superabsorbent particles per cm³ of the liquid-spreading and storage layer 12 is preferably smaller than 1100. According to a preferred embodiment, the average absorption capacity per superabsorbent particle in the liquid-distribution and storage layer 12 is greater than 9.5 mg, and the number of superabsorbent particles per cm³ of the liquid-distribution and storage layer is smaller than 600. According to a further example, the average absorption capacity per superabsorbent particle in the liquid-distribution and storage layer 12 is greater than 14.0 mg, and the number of superabsorbent particles per cm³ of the liquid-distribution and storage layer is smaller than 450. The absorption capacity is measured throughout using 0.9% by weight sodium chloride solution.

The average distance between centres of the superabsorbent particles in the liquid-distribution and storage layer 12 in a dry state is, for example, greater than 700 micrometres, preferably greater than 1000micrometres and even more preferably greater than 1200 micrometres. The density of the absorbent structure in the liquid-distribution and storage layer 12 is, for example, greater than 0.12 g/cm³, preferably greater than 0.17 g/cm³ and even more preferably greater than 0.25 g/cm³.

Suitable materials for use as the receiving layer 11 include, for example, an open nonwoven layer made of synthetic or natural fibres. A difference in properties between the liquid-distribution and storage layer 12 and the receiving layer 11 can be brought about by, for example, the liquid-distribution and storage layer 12 being compressed more firmly than the receiving layer 11. A firmly compressed fibrous structure with high density spreads the liquid better than a corresponding fibrous structure with lower density, which, because of its larger pore size, has a higher instantaneous liquid absorption capacity but lower wicking capacity. Differences in absorption properties between the two layers can also be brought about by means of different fibrous structures with different properties. Accordingly, cellulose fluff pulp produced in a conventional chemical way, chemical pulp (CP), has higher liquid-wicking capacity compared with pulp produced in a mechanical or chemithermomechanical way. Therefore, cellulose fluff pulp produced in a conventional chemical way, chemical pulp (CP), is an example of a suitable material for the liquid-distribution and storage layer 12, and pulp produced in a mechanical or chemithermomechanical way is an example of a material for the receiving layer 11. A fibrous structure containing chemically stiffened cellulose fibres also has a higher instantaneous liquid absorption capacity but lower spreading capacity than conventional chemical pulp and is therefore an example of a material for the receiving layer 11. Another suitable material for use as the receiving layer 11 is a superabsorbent foam, for example a polyacrylate-based foam. A polyacrylate-based foam is produced by a solution which includes at least monomer, cross-linker, initiator and surfactant being saturated and pressurized with carbon dioxide in a vessel while being stirred. When the solution is removed from the vessel through a nozzle, the solution expands and a foamed structure is obtained. The foamed structure is then locked by polymerization and cross-linking initiated by, for example, UV radiation. Finally, the material is compressed and dried. On wetting, such a superabsorbent foam expands greatly, which results in it being capable of receiving a large quantity of liquid in a short time. Such a receiving layer may comprise, for example, a continuous layer, which is positioned at least in the crotch portion of the article, or alternatively of a number of strips with hollow spaces between the strips. The receiving layer can also include a fibrous layer with superabsorbent particles or a superabsorbent coating bonded to the fibrous layer.

In order to reduce the occurrence of undesirable bacterial growth and problems with odour, the absorbent structure 3 and/or the liquid-permeable surface layer 1 can comprise bacteria-inhibiting and/or odour-inhibiting substances. An example of a bacteria-inhibiting and odour-inhibiting substance is a superabsorbent material which has a lower pH than a conventional superabsorbent. A superabsorbent material with a lower pH than a conventional superabsorbent has a lower degree of neutralization than a conventional superabsorbent, the degree of neutralization being, for example, between 20 and 60%. The superabsorbent particles according to the invention can be, for example, a superabsorbent with a degree of neutralization between 20 and 60%.

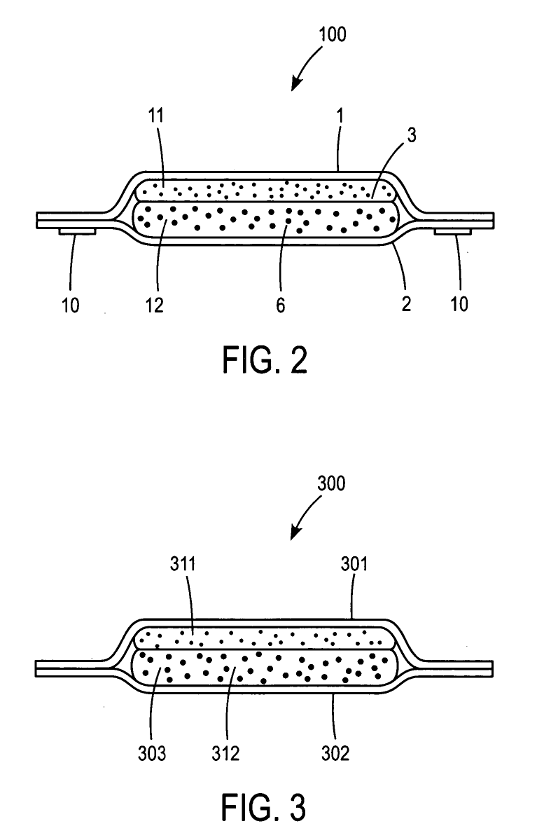

FIG. 2 shows a cross section along the line II-II through the diaper 100 shown in FIG. 1. The diaper 100 shown in FIG. 2 therefore has a liquid-permeable surface layer 1, a backing layer 2, and an absorbent structure 3 enclosed between the liquid-permeable surface layer 1 and the backing layer 2.

The absorbent structure 3 of the diaper is constructed in a preferred embodiment from an upper liquid-receiving layer 11 and a lower liquid-distribution and storage layer 12. The lower liquid-distribution and storage layer 12 in the absorbent structure 3 includes an absorbent layer according to the invention. The liquid-distribution and storage layer 12 therefore also comprises superabsorbent particles in addition to cellulose fluff pulp. The average absorption capacity per superabsorbent particle in the liquid-spreading and storage layer 12 is greater than 8.0 mg sodium chloride solution, and the number of superabsorbent particles per cm³ of the liquid-spreading and storage layer 12 is smaller than 1100.

FIG. 3 shows a cross section of an alternative embodiment of an absorbent article according to the invention. The diaper 300 shown in FIG. 3 is essentially constructed in the same way as the diaper in FIG. 2. The diaper 300 therefore has a liquid-permeable surface layer 301, a backing layer 302, and an absorbent structure 303 enclosed between the liquid-permeable surface layer 301 and the backing layer 302.

The absorbent structure 303 of the diaper is constructed from an upper liquid-receiving layer 311 and a lower liquid-distribution and storage layer 312. Both the upper liquid-receiving layer 311 and the lower liquid-distribution and storage layer 312 in the absorbent structure 303 include absorbent layers according to the invention. The upper liquid-receiving layer 311 and the lower liquid-distribution and storage layer 312 therefore have fibrous structures which comprise superabsorbent particles, the average absorption capacity per superabsorbent particle in both layers 311, 312 preferably being greater than 8.0 mg sodium chloride solution. Furthermore, the number of superabsorbent particles per cm³ of the absorbent structure in both layers is preferably smaller than 1100. In the absorbent structure 303, both the upper liquid-receiving layer 311 and the lower liquid-distribution and storage layer 312 therefore include absorbent layers according to the invention.

The invention is of course not limited to the illustrative embodiments above but can of course be applied to other embodiments within the scope of the patent claims. The invention therefore also comprises incontinence pads, pant diapers, sanitary towels, panty liners and the like. The invention also includes belt-supported diapers.

It is furthermore possible, for example, for the entire the absorbent structure to have only one absorbent layer, in which case the entire absorbent structure comprises only an absorbent layer according to the invention. According to another example, the absorbent structure can include a multilayer structure where the upper liquid-receiving layer includes an absorbent layer according to the invention. The liquid-receiving layer therefore comprises fibres and superabsorbent particles, the average absorption capacity for the superabsorbent particles in the liquid-receiving layer preferably being greater than 8.0 mg sodium chloride solution (% by weight of sodium chloride is 0.9%), and the number of superabsorbent particles per cm³ of the liquid-receiving layer preferably being smaller than 1100. A cellulose fluff pulp with superabsorbent material of conventional type, for example, is used as the liquid-distribution and storage layer. It is also possible for the liquid-distribution and storage layer to include several different layers, at least one of the layers containing superabsorbent material and, for example, one layer being formed of pure pulp in order to obtain good liquid distribution. Different layers can moreover have a difference in concentration of superabsorbent material, a liquid-distribution and storage layer with a gradually increasing/decreasing content of superabsorbent material being obtained. It is also possible for the liquid-distribution and storage layer to comprise a layer made of a superabsorbent foam.

It is furthermore also possible for the absorbent structure to include one or more tissue layers or types of material or component other than those described above. The design of the layers can also vary. For example, one or more layers in the absorbent structure can have cut-outs, that is to say cavities. The cut-outs extend, for example, in the longitudinal direction of the absorption structure. It is of course also possible to have other physical designs of the cut-outs.

### EXAMPLE 1

### Determining Volume and Density of the Absorbent Layer

When measuring the volume (cm³) of the absorbent layer in an absorbent article, the absorbent layer is separated from the rest of the material in the article. If the absorbent structure has several different absorbent layers with mutually different properties, the various absorbent layers are also separated from one another, after which volume and density are measured for each absorbent layer.

The absorbent layer is then weighed, and the thickness of the absorbent layer is measured. When measuring the thickness, use is made of a thickness gauge which has a circular foot with a diameter of 80 mm. The foot is to exert a pressure of 0.5 kPa on the absorbent layer. The thickness is measured at five different points, which are distributed uniformly over the surface of the absorbent layer. The average value from these five measuring points represents the thickness of the absorbent layer in the volume calculation. The area of the absorbent layer is then measured, the volume being obtained by multiplying the thickness by the area. The density of the absorbent layer is then obtained by dividing the weight of the absorbent layer by the volume.

### EXAMPLE 2

### Determining the Number of Superabsorbent Particles per Unit of Volume and Measuring the Absorption Capacity of the Superabsorbent Particles

The example is based on an absorbent layer which contains the superabsorbent particles. In this connection, it is also described how the superabsorbent particles are to be separated from the pulp structure. Since it is important than no material is lost in the handling described below, measures should be taken to avoid loss of material.

The absorbent layer is first separated from the rest of the material in the article. The superabsorbent particles are then separated from the fluff pulp in the absorbent layer by finely dividing the layer, that is to say tearing it into small pieces, and then shaking the superabsorbent particles out of the pulp structure. It is also possible to use an apparatus for separating the superabsorbent particles from the pulp structure. If an apparatus is used for separating the superabsorbent particles from the pulp structure, however, it is a condition that the superabsorbent particles are not damaged mechanically. The moisture content of the superabsorbent particles should be less than 5.0%. All indications in the present invention relate to superabsorbent particles with a moisture content of less than 5.0%. The moisture content is determined according to the method ISO 17190-4 “Determination of moisture content by mass loss upon heating”. If the moisture content exceeds 5.0%, the superabsorbent is dried at 60° C. until the moisture content is less than 5.01%.

Particles with a diameter smaller than 150 μm are then separated in Example 2. All indications of the number of superabsorbent particles in Example 2 relate to particles with a diameter of 150 μm or greater. Particles with a diameter smaller than 150 μm are therefore not included in the expression “superabsorbent particles” according to Example 2 given above. In order to separate particles smaller than 150 μm, use is made of apparatus described in ISO 17190-3 “Determination of particle size distribution by sieve fractionation”. Particles smaller than 150 μm are sieved out. The remaining particles are then weighed. This weight therefore constitutes the total weight of the superabsorbent particles for Example 2.

In a preferred embodiment: of the invention, particles with a diameter smaller than 50 μm are separated out and the calculations regarding the number of superabsorbent particles in the preferred embodiment will relate to particles with a diameter of 50 μm or greater. Particles with a diameter smaller than 50 μm are therefore not included in the calculations according to the preferred embodiment. In order to separate particles smaller than 50 μm, use is made of apparatus described in ISO 17190-3 “Determination of particle size distribution by sieve fractionation”. Particles smaller than 50 μm are sieved out. The remaining particles are then weighed. This weight constitutes the total weight of the superabsorbent particles in the preferred embodiment. It has been found that basing the calculations on superabsorbent particles having a diameter of 50 μm or greater is preferred for being able to obtain the desired results while still excluding the inconsequential addition of superabsorbent particle dust.

In order to calculate the number of superabsorbent particles per unit of volume, the superabsorbent particles are divided up into smaller portions. To divide the superabsorbent particles up into smaller portions, use is made of a “Rotary Sample Divider—laborette 27” from Fritsch GmbH Laborgerätebau or similar apparatus. Each portion is assumed to have a representative particle size distribution. Three of these portions are then weighed, and the number of particles in these three portions is counted manually. Each portion weighed 0.1 gram, so altogether the number of superabsorbent particles in 0.3 gram was counted. The weight of the samples is to be +/−10% of the given values. The measuring accuracy is to be +/−0.005 gram. The average weight of the individual superabsorbent particles is then calculated by dividing the weight of the sample (roughly 0.3 gram) by the number of manually counted particles. By then dividing the total weight of the superabsorbent particles by the average weight of the superabsorbent particles, the total number of superabsorbent particles in the absorbent layer is obtained. In order finally to obtain the number of superabsorbent particles per cm³ of the absorbent layer, the total number of superabsorbent particles is divided by the volume of the absorbent layer.

The absorption capacity of the superabsorbent particles is then measured according to ISO 17190-6 “Gravimetric determination of fluid retention after centrifugation”. The absorption capacity is measured on three other portions. All portions have a representative particle size distribution, and the absorption capacity per particle can therefore be calculated by dividing the measured absorption capacity by the number of particles previously counted manually. Liquid used for measurement is 0.9% by weight sodium chloride solution.

Superabsorbent materials tested are three different size fractions of particulate polyacrylate-based superabsorbent from BASF with the designation Hysorb C 7100 and two different size fractions of particulate polyacrylate-based superabsorbent from Dow with the designation Drytech S230R. The average particle size of the superabsorbent particles from BASF with the designation Hysorb C 7100 was in the first case a normal particle-size distribution, that is to say the measurement was performed on the whole particle fraction in the commercially available grade; in the second case the particle size was between 600 μm and 710 μm, and in the third case the average particle size was between 710 μm and 850 μm. The average particle size of the superabsorbent particles from Dow with the designation Drytech S230R was in one case a normal particle size distribution, that is to say the measurement was performed on the whole particle fraction in the commercially available grade, and the average particle size in the other case was greater than 600 μm.

The superabsorbent with the designation Hysorb C 7100 with a normal particle size distribution is called A below.

The superabsorbent with the designation Hysorb C 7100 with a particle size between 600 μm and 710 μm is called B below.

The superabsorbent with the designation Hysorb C 7100 with a particle size between 710 μm and 850 μm is called C below.

The superabsorbent with the designation Drytech S230R with a normal particle size distribution is called D below.

The superabsorbent Drytech S230R with a particle size greater than 600 μm is called E below.

Result

| Superabsorbent | Abs. cap. (g/g) | Abs. cap/particle (mg/particle) |
|---|---|---|
| A | 35.6 | 1.4 |
| B | 38.0 | 7.4 |
| C | 37.2 | 9.5 |
| D | 33.6 | 1.7 |
| E | 36.5 | 8.4 |

It can be seen from the result that the average absorption capacity per particle for superabsorbent C and E is greater than 8.0 mg sodium chloride solution, while the average absorption capacity per particle for superabsorbent A, B and D is less than 8.0 mg sodium chloride solution.

### EXAMPLE 3

### Measuring Admission Time in Absorbent Structure

Measurement of admission time on a first, a second, a third and a fourth measurement was performed for five different absorbent structures. The absorbent structures contained 50% by weight superabsorbent and 50% by weight chemical fluff pulp. The chemical fluff pulp was manufactured by Weyerhauser and is called NB 416.

Superabsorbent material in absorbent structure 1 was superabsorbent A, that is to say Hysorb C 7100 with a normal particle size distribution.

Superabsorbent material in absorbent structure 2 was superabsorbent B, that is to say Hysorb C 7100 with a particle size between 600 μm and 710 μm.

Superabsorbent material in absorbent structure 3 was superabsorbent C, that is to say Hysorb C 7100 with a particle size between 710 μm and 850 μm.

Superabsorbent material in absorbent structure 4 was superabsorbent D, that is to say Drytech S230R with a normal particle size distribution.

Superabsorbent material in absorbent structure 5 was superabsorbent E, that is to say Drytech S230R with a particle size greater than 600 μm.

The absorbent structures 1, 2 and 3 had a density which was 0.25 g/cm³, a weight per unit area which was 600 g/m² and an area which was 10×28 cm.

The absorbent structures 4 and 5 had a density which was 0.25 g/cm³, a weight per unit area which was 600 g/m2 and an area which was 10×40 cm.

For measurement, the absorbent structure was placed on a foam mattress of the tempur type. The absorbent structure was then subjected to a load of 0.64 kPa and four doses of 80 ml each of sodium chloride solution (0.9% by weight) were added. The time between the liquid doses was 10 minutes. The time for the liquid to be admitted into the absorbent structure was measured. The admission time was measured in seconds:

Result

| | Abs. | Abs. | | | Abs. |
|---|---|---|---|---|---|
| | struct. | struct. | Abs. | Abs. | struct. |
| | 1 (s) | 2 (s) | struct. 3 (s) | struct. 4 (s) | 5 (s) |
| 1st wetting | 115 | 103 | 110 | 67 | 83 |
| 2nd wetting | 210 | 149 | 146 | 80 | 69 |
| 3rd wetting | 312 | 230 | 217 | 122 | 109 |
| 4th wetting | 354 | 275 | 259 | 160 | 132 |

The result shows that of the absorbent structures which contained superabsorbent Hysorb C 7100, that is to say absorbent structures 1-3, absorbent structure 3 has the fastest admission time on repeated wetting. Absorbent structure 3 contained superabsorbent particles with an average absorption capacity which is greater than 8.0 mg.

Of the absorbent structures which contained Drytech S230R, that is to say absorbent structures 4-5, absorbent structure 5 has the fastest admission time on repeated wetting. Absorbent structure 5 contained superabsorbent particles with an average absorption capacity which is greater than 8.0 mg, while absorbent structure 4 contained superabsorbent particles with an average absorption capacity which is lower than 8.0 mg.

### EXAMPLE 4

### Measuring Liquid Distribution in Absorbent Structure

Measurement of liquid distribution after a first, a second, a third and a fourth wetting was performed for absorbent structure 4 and absorbent structure 5. The liquid distribution was measured after each wetting immediately before the next liquid dose was added. The liquid distribution was measured in cm.

Result

| | Abs. struct. 4 (cm) | Abs. struct. 5 (cm) |
|---|---|---|
| 1st wetting | 20 | 25 |
| 2nd wetting | 22 | 26 |
| 3rd wetting | 29 | 34 |
| 4th wetting | 34 | 39 |

The result shows that absorbent structure 5 which contained superabsorbent particles with an average absorption capacity which is greater than 8.0 mg spreads the liquid further than absorbent structure 4 which contained superabsorbent particles with an average absorption capacity which is lower than 8.0 mg.

### EXAMPLE 5

### Measuring Rewet

Measurement of rewet after the fourth wetting was performed for absorbent structure 4 and absorbent structure 5. Measurement of rewet was started 10 minutes after the fourth dose of liquid had been applied. After 10 minutes, 15 pieces of filter paper and a weight (5 kPa) were placed on the wetting point of the absorbent structure. After 15 seconds, the weight was removed, and the bundle of filter paper was weighed. The rewet was calculated by subtracting the dry weight of the filter paper from the wet weight. The rewet was measured in grams of sodium chloride solution (0.9% by weight).

Result

Absorbent structure 4: 9.5 grams

Absorbent structure 5: 7.9 grams

The result shows that absorbent structure 5 has lower rewet than absorbent structure 4. The result therefore shows that the absorbent structure which contained superabsorbent particles with an average absorption capacity which is greater than 8.0 mg has lower rewet than the absorbent structure which contained superabsorbent particles with an average absorption capacity which is lower than 8.0 mg.

The present invention also includes all conceivable combinations of the preferred embodiments described herein. Moreover, the invention is not limited to the above-mentioned preferred embodiments, or the construction of the exemplary diaper above, and instead it can of course be applied to other embodiments within the scope of the attached patent claims.

Although only preferred embodiments are specifically illustrated and described herein, it will be appreciated that many modifications and variations of the present invention are possible in light of the above teachings and within the purview of the appended claims without departing from the spirit and intended scope of the invention.

What is claimed is:

1. An absorbent structure for use in an absorbent article, said absorbent structure comprising:
at least one absorbent layer comprising fluff pulp and superabsorbent particles uniformly dispersed throughout, wherein the superabsorbent particles have a particle size which is greater than 50 μm, wherein an average absorption capacity per superabsorbent particle in the absorbent layer is greater than 8.0 mg sodium chloride solution and the number of uniformly dispersed superabsorbent particles per cm³ of the absorbent layer is smaller than 1100.

2. An absorbent structure according to claim 1, wherein the average absorption capacity per superabsorbent particle in the absorbent layer is greater than 9.5 mg sodium chloride solution and the number of superabsorbent particles per cm³ of the absorbent layer is smaller than 600.

3. An absorbent structure according to claim 2, wherein the average absorption capacity per superabsorbent particle in the absorbent layer is greater than 14.0 mg sodium chloride solution and the number of superabsorbent particles per cm³ of the absorbent layer is smaller than 450.

4. An absorbent structure according to claim 1, wherein the superabsorbent particles have a particle size which is greater than 600 μm.

5. An absorbent structure according to claim 1, wherein an average distance between centers of adjacent said superabsorbent particles in the absorbent layer in a dry state is greater than 700 μm.

6. An absorbent structure according to claim 5, wherein the average distance between centers of adjacent said superabsorbent particles in the absorbent layer in a dry state is greater than 1000 μm.

7. An absorbent structure according to claim 6, wherein the average distance between centers of adjacent said superabsorbent particles in the absorbent layer in a dry state is greater than 1200 μm.

8. An absorbent structure according to claim 1, wherein a density of the absorbent layer in a dry state is greater than 0.12 g/cm³.

9. An absorbent structure according to claim 8, wherein the density of the absorbent layer in a dry state is greater than 0.17 g/cm³.

10. An absorbent structure according to claim 9, wherein the density of the absorbent layer in a dry state is greater than 0.25 g/cm³.

11. An absorbent structure according to claim 1, wherein the at least one absorbent layer further comprises a bonding means.

12. An absorbent structure according to claim 1, wherein the superabsorbent particles are surface cross-linked.

13. An absorbent article comprising the absorbent structure according to claim 1.
